(19) United States
(12) Patent Application Publication
(10) Pub. No.: US 2024/0393350 A1
(43) Pub. Date: Nov. 28, 2024

(54) METHOD FOR THE EARLY DIAGNOSIS OF NEURODEGENERATIVE DISEASES BY MEANS OF QUANTIFICATION OF PRONGF AND DERIVED FORMS THEREOF

(71) Applicant: EUROPEAN BRAIN RESEARCH INSTITUTE (EBRI) R.LEVI MONTALCINI, ROMA (RM) (IT)

(72) Inventors: Antonino CATTANEO, ROMA (RM) (IT); Francesca MALERBA, ROMA (RM) (IT)

(21) Appl. No.: 18/698,716

(22) PCT Filed: Oct. 3, 2022
(86) PCT No.: PCT/IB2022/059416

§ 371 Date: Apr. 4, 2024

(30) Foreign Application Priority Data
Oct. 7, 2021 (IT) 102021000025619

(51) Int. Cl. G01N 33/68

(52) U.S. Cl. CPC G01N 33/6896; G01N 2333/48; G01N 2800/2814; G01N 2800/2821

(57) ABSTRACT

Method for the early diagnosis and the monitoring of the evolution/regression of neurodegenerative pathologies, said method providing for the quantification of a, biomarker for said pathologies in a fluid that was previously drawn from a, patient, said fluid being selected from among: cerebrospinal fluid; serum; urine; post mortem cerebral tissues and cellular lysates, said method being characterized in that the quantified biomarker is selected from among native proNGF; modified proNGF, the latter being proNGF in its forms with higher molecular weight, including forms of 39-40 kDa and 45-50 kDa; NGF; and the proNGF/NGF ratio, said method sequentially providing for the following steps of Preparation of the biological sample. Definition of the calibration curve: Execution of run and interpolation.

### FIELD OF THE ART

The present invention refers to the medical field and to the biotechnology field. More in detail the present invention regards a particular and innovative method for the early diagnosis of neurodegenerative diseases such as, as a non-limiting example, Alzheimer's Disease and for the stratification of subgroups of patients. Said method is based on the quantification of specific biomarkers in biological fluids that were previously drawn from patients having said pathologies, even in their initial stages.

### Prior Art

The role of NGF, and of its proNGF precursor, is widely known in Alzheimer's Disease, as therapeutic and diagnostic target.

The nerve growth factor (NGF), the first neurotrophin discovered by Rita Levi-Montalcini, acts on the peripheral and central nervous system and is responsible for the correct development of the nervous system during adult and embryonic life. NGF is produced within the cell as a precursor, proNGF, which is cut by the furin protease in the endoplasmic reticule to give the mature form.

It is by now known that proNGF is not only a mere precursor, but also performs distinct functions with respect to its mature counterpart (Hempstead, 2006). The two neurotrophins, in fact, by acting on different receptors, can give rise to a neurotrophic signal due to NGF action; on the other hand, the proNGF can induce cellular death. It is the end balance between the levels of NGF and proNGF that ensures the correct operation of the central nervous system in its physiological conditions.

It has in fact been demonstrated by various research groups that, in the adult brain, an imbalance of proNGF with respect to NGF is associated with neurodegeneration, and that the levels of proNGF are increased in the brain of subjects affected by Alzheimer's and in animal models of the disease, with respect to healthy controls (Capsoni et al., 2011, 2010; Capsoni and Cattaneo, 2006; Counts and Mufson, 2005; Iulita and Cuello, 2014; Pentz et al., 2020; Tiveron et al., 2013) ProNGF has however never been measured, since there no assays on the market that are capable of measuring the proNGF in tissues, as the same datasheets of the commercial kits state, and as has been demonstrated (Malerba et al., 2016).

The reason for which the proNGF has up to now never been measured is both due to the biochemical characteristics of flexibility of the pro-peptide (Paoletti et al., 2011, 2009) which complicate the obtainment of antibodies with high affinity, and since NGF and proNGF, if present simultaneously in a sample, reciprocally interfere, altering and thus making the measurement result unreliable. (Malerba et al., 2016). The problem is non-negligible since numerous biological samples contain both forms, therefore the only way to obtain reliable measurements of the two neurotrophins is to separate them and measure them separately. Up to now the only way to evaluate the relative levels of proNGF with respect to NGF was in fact Western Blot, or protein precipitation and Western Blot (Counts and Mufson, 2005; E Counts et al., 2016; Pentz et al., 2020; Tiveron et al., 2013).

The proNGF thus represents a potential biomarker of neurodegeneration, even if up to now it was not possible to measure the concentration thereof in biological fluids of clinical importance.

In diseases such as Alzheimer's, whose diagnosis, and in particular early diagnosis is still very difficult, and in which therapeutic intervention would be necessary in the initial stages of the disease, finding valid and easily measurable biomarkers is an urgent necessity. In addition, Alzheimer's Disease covers a very heterogeneous spectrum of patients and a prediction medicine would benefit from the possibility to stratify the patients based on biomarkers. Therefore, being able to measure the levels of proNGF in the CSF or in other biological fluids is a subject of great interest in the field of clinical research (E Counts et al., 2016; Iulita and Cuello, 2014). In the literature, up to now, in only one paper, the proNGF was detected in human ventricular CSF of post mortem patients (40 patients and 24 controls), by means of semiquantitative western blot, without calibration curve (E Counts et al., 2016).

In light of that expressed above, experimental proof with regard to the role that the proNGF takes on in neurodegeneration—and consequently in the possible determination of the progression of neurodegenerative pathologies—the present invention proposes a new and effective method that, overcoming all the aforesaid critical issues associated with the quantification of the proNGF, allows obtaining a strong and reliable determination of its levels, as well as the attainment of all the advantages associated with the definition of the extent of the neurodegenerative pathology in its various development stages and with the direct monitoring of the effectiveness of specific therapeutic treatments.

### DESCRIPTION OF THE INVENTION

The present description refers to a new method for defining the various stages of neurodegenerative pathologies, typically but not limited thereto of Alzheimer's Disease, which is based on the quantification of the levels of a specific biomarker, the proNGF, and of derived forms thereof, in fluids previously drawn from patients having evident or suspect signs that are symptomatic with said pathologies, but also in asymptomatic patients, as preventative screening.

More in detail, the method according to the present invention allows measuring proNGF, preferably in the CSF, of patients with initial or light forms of dementia.

Advantageously said measurement is of considerable medical importance for the early diagnosis of Alzheimer's Disease;

Advantageously said measurement also allows an improved stratification of the patients which translates into an improved therapeutic intervention;

Advantageously said measurement also allows following the progression of the proNGF, during an experimental therapy, so as to monitor the effectiveness of the treatment itself.

The method for monitoring the extent and the evolution of neurodegenerative diseases according to the present invention therefore meets the urgent need of providing a simple, reliable and automated measurement for the attainment of all the abovementioned advantages. For such purpose the inventors have designed a method capable of measuring the concentration of proNGF in the CSF. The method makes use of capillary electrophoresis, for example in an exemplifying but non-exhaustive manner, using the Simple Wes (Protein Simple) instrument which is automatic and processes 25 run samples. Being based on capillary electrophoresis in naturing conditions, the system separates the proNGF from NGF by molecular weight, thus preventing the reciprocal interference. The method is divided into three separate passages.

Still more in detail, the inventors have designed a method for measuring the proNGF in the human CSF which consists of at least three steps that provide for the following actions in sequence:
preparation of the biological sample;
calibration curve;
run and interpolation.

The aforesaid and further advantages obtainable with the present method will be made more appreciable in the following description.

Before commencing the following detailed description, it is of interest to specify that biomarkers, according to an FDA classification (Molinuevo et al., 2018), can be of different types, based on their use:
Diagnostic: they allow a precise diagnosis of the pathology, and a differential diagnosis with respect to other diseases with similar phenotype;
Monitoring: they allow evaluating the stage of progression of the disease;
Predictive: they can indicate the future evolution of the disease;
Prognostic: they allow the stratification of the patients (and consequently if they fall within the criteria of inclusion or exclusion in case of clinical experimentation);
Pharmacodynamics: they allow following the progression of the pathology following a treatment.

The proNGF is undoubtedly a diagnostic, monitoring and prognostic biomarker and the studies conducted during the definition of the method according to the present invention indicate that it could also be predictive and pharmacodynamic on the basis of a rational.

More in detail the studies conducted by the Applicant have shown that the pathologies for which the proNGF is a biomarker are:
5-Alzheimer's Disease, on the basis of: strong rationale, numerous proof in the literature (Cuello et al., 2019; E Counts et al., 2016; Fahnestock et al., 2001; Fahnestock and Shekari, 2019; Pentz et al., 2020); and it was demonstrated by the present method.
Down's Syndrome: strong rationale; proof in the literature (Pentz et al., 2021, 2019);
Frontotemporal Dementia: proof in the literature (Belrose et al., 2014);
Multiple Sclerosis: proof in the literature (Acosta et al., 2013; Zhang et al., 2009; Zhu et al., 2016);
Amyotrophic lateral sclerosis: behavior similar to the samples of Alzheimer's Disease;
Parkinson's Disease and Parkinsonism: proof in the literature (Xu et al., 2018);
Chronic pain: strong rationale.
   The Peaks of proNGF Visible in the Pherogram

Nearly all the measured samples of cerebrospinal fluid (CSF) contain, in addition to the measured peak of proNGF, with molecular weight of 34 KDa, corresponding to the “nude” form of proNGF, lacking post-translational modifications, other two peaks with greater molecular weight (39 KDa and 45-50 KDa) also identified as proNGF, not only by means of the present method, but also with Western Blot and with Mass Spectrometry. These forms or similar forms with higher molecular weight have already been described in the literature and it was assumed that they are proNGF with post-translational modifications (e.g. glycosylations, lipoxylations, etc.). Proof from the literature also shows that such forms with high molecular weight increase in Alzheimer's Disease. (Kichev et al., 2009; Pedraza et al., 2005)

On the basis of a rationale and of the literature, the inventors have also assumed that the diminution of the proNGF to 34 KDa, observed with said method for Alzheimer patients with respect to the controls, can be due to a conversion of the proNGF form to 34 KDa to the forms with higher molecular weight.

In an entirely unexpected manner, most of the samples measured also show the presence of a peak corresponding to mature NGF. The result was not expected since from the literature it is known that physiologically NGF is much less concentrated than proNGF, and it is very difficult not only to measure it, but even to find it. (Fahnestock et al., 2001) This aspect is an important and significant indication of the sensitivity of the method.

Indeed, the NGF peak areas are much lower than the proNGF peak areas, and sometimes below the signal/noise value that is deemed reliable. This lower abundance of the NGF peaks require that the measurement is optimized for the NGF peak. It was not possible to measure NGF in all the samples, but only for a small number of these.

In addition, as demonstrated by the authors of the present invention in preceding publications (Malerba et al., 2016), the measurement of NGF in samples containing both the mature form NGF and the precursor proNGF is technically incorrect, if carried out in native conditions, with anti-NGF antibodies. In order to obtain a reliable measurement of NGF, it is necessary to work in denaturing conditions, and separate the proNGF from NGF, exactly as occurs with the present method.

The method developed by the Applicant is such to ensure that the diagnostic result can be reached by electing as biomarker, as one wishes: proNGF, intending in addition to the native form at 34 kDa, also its modified forms corresponding to the peaks at 39-40 kDa and 45-50 kDa; NGF; and the measurement of the proNGF/NGF ratio. The latter, in particular, represents a biomarker with a strong rationale in the literature, and probably more effective than the pure measurement of proNGF. With the present method, following optimization, the measurement of proNGF/NGF could occur in the same sample, in a multiplex manner, and be reliable so that the technical interference described in Malerba et al 2016 would not be present. (Malerba et al., 2016).

As described above, the method according to the present invention was validated in cerebrospinal fluid, but it is taken for granted that the person skilled in the art understands that its applicability is also extended to serum, urine, post-mortem cerebral tissues and cellular lysates.

### DESCRIPTION OF THE FIGURES

The invention will be described in detail hereinbelow, also with reference to the enclosed figures in which:

FIG. 1 shows the calibration curve of the sample.

FIG. 2 shows the curves relative to three samples representative of CSF of different Alzheimer patients.

FIG. 3 shows the curves relative to the experiment of deprivation with immunoprecipitation.

FIG. 4 shows the results relative to the experiment of mass spectrometry: Gel SDA-PAGE (FIG. 4(A)) and results: Beta-NGF (FIG. 4 (B) and % Beta-NGF FIG. 4 (C)).

FIG. 5 (A) shows the calibration curves of samples of CSF with a high concentration of proNGF, run as is and concentrated.

FIG. 5 (B) shows the analysis of the peaks.

FIG. 5 (C) shows further analysis of the peaks.

FIG. 5 (D) shows further analysis of the peaks.

FIG. 6 shows the calibration curves in independent experiments.

FIG. 7 shows the curves relative to the cross-reactivity experiment.

### DETAILED DESCRIPTION OF THE INVENTION

The method for measuring the proNGF in human CSF consists of at least three steps that provide for the following actions in sequence:
preparation of the biological sample;
calibration curve;
run and interpolation

More in detail:

### Preparation of the Biological Sample

The CSF must be concentrated so that the proNGF can be correctly detected and measured in the dynamic range of the calibration curve. In addition, the CSF must be desalted. The natural high ionic force of the CSF is in fact incompatible with the electrophoretic run.

The CSF is desalted by means of a disposable desalting column (Zeba Spin). The protein precipitation is instead executed by means of TCA, the sample is then resuspended in 0.1× Simple Wes Buffer, resulting 13 times concentrated.

The precipitation with TCA, in addition to having the advantage of concentrating the sample, denatures all the proteins, an advantageous process also from the biological safety standpoint. The sample is then admixed with the Master Mix Simple Wes and with 0.1 M DTT, boiled for 5 minutes, aliquoted and preserved at −80° C.

### Calibration Curve

The calibration curve is obtained with 8 serial dilutions (1:2) of recombinant human proNGF.

The dynamic range is 4000 ng/ml-31 ng/ml.

The points of the curve are prepared once a month, admixed with Master Mix Simple Wes and 0.1 M DTT, boiled for 5 minutes, aliquoted and preserved at −80° C. Each month a batch to batch consistency control is executed between the new and the old curve.

### Run and Interpolation

The Simple Wes is an automated instrument, based on a capillary electrophoresis in denaturing conditions, which mimics the electrophoretic run on gel. The proteins are then recognized by primary antibodies, and the signal amplified with secondary antibodies conjugated with peroxidase. One can increase the amplification of the signal, using biotinylated secondary antibodies and streptavidin bonded to the HRP enzyme. With respect to the Western Blot, it is possible to quantify the sample with improved reliability, waste less reagents (only 5 microliters per capillary) and less time. It is also more sensitive than a Western Blot.

The Simple WES run of the method developed by the Applicant is executed on 2-40 KDa cartridges with the parameters described in table 1, which we validated. The primary antibody used is anti NGF MyBiosource cod. MBS125020 (1:50), the secondary is biotinylated Jackson anti rabbit with low cross reactivity (1:100). The calibration curve is run together with the duplicates of each sample.

TABLE 1
run of the samples

| Separation matrix | |
|---|---|
| Stacking matrix | |
| Position | M1 |
| Loading time (s) | 14 |
| Sample | |
| Position | A1 |
| Loading time (s) | 11 |
| Separation time (min) | 27 |
| Separation voltage (volts) | 375 |
| Incubation time of the antibody diluent (min) | 5 |
| Incubation time of the primary antibody (min) | 90 |
| Incubation time of the secondary antibody (min) | 30 |
| Incubation time of the tertiary antibody (min) | 30 |

The samples of CSF are run at least 4 times, 2 duplicated in two different runs.

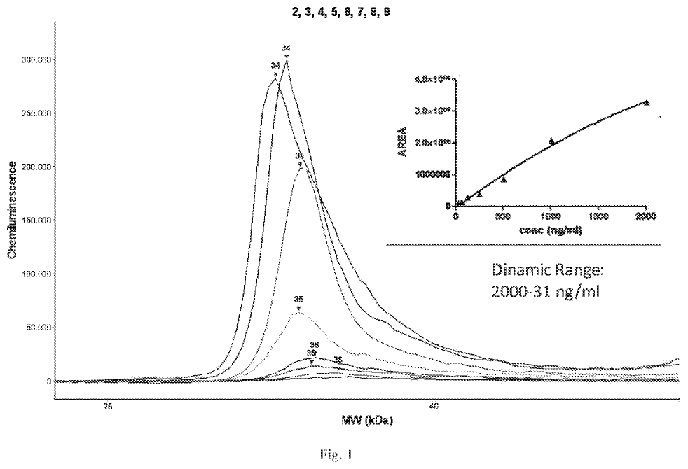

The peaks obtained as output from the Simple WES are interpolated by means of the program Compass. The area of the peak is proportional to the concentration of the protein. The calibration curve is interpolated with a polynomial equation of order 2 by the program GraphPAD Prism. The value of concentration of the samples is obtained by interpolating the value of the area of the peak corresponding to the proNGF with the calibration curve. (FIG. 1).

The method was validated by means of samples of human CSF of neurodegenerative diseases and controls.

### Results

The proNGF in the calibration curve gives rise to peaks with molecular weight of 34 kDa. (FIG. 1).

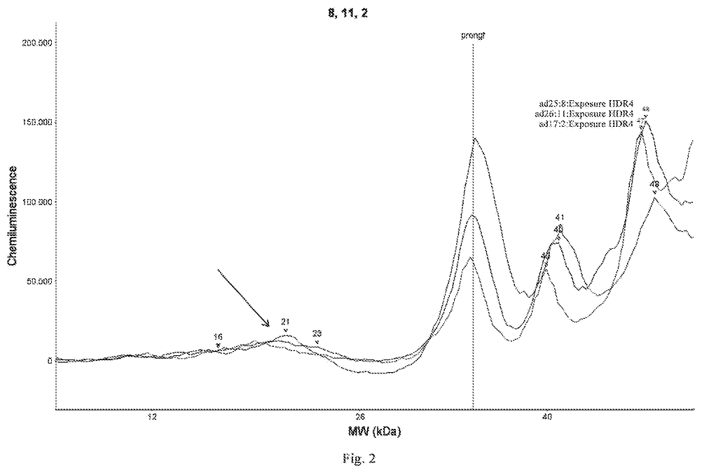

The samples of CSF instead have three peaks with different relative height. The peaks correspond to the following molecular weights: 34 KDa, 39-40 KDa, 45-50 KDa. Surprisingly, some samples have, in addition to the described three peaks, also another peak of small dimensions at the height of 18-20 KDa, corresponding to the molecular weight of mature NGF, indicated by the arrow. (FIG. 2).

In order to understand the specificity of these peaks, detected by the anti-polyclonal antibody anti NGF MyBiosource, a deprivation of the biological sample was carried out. A sample of

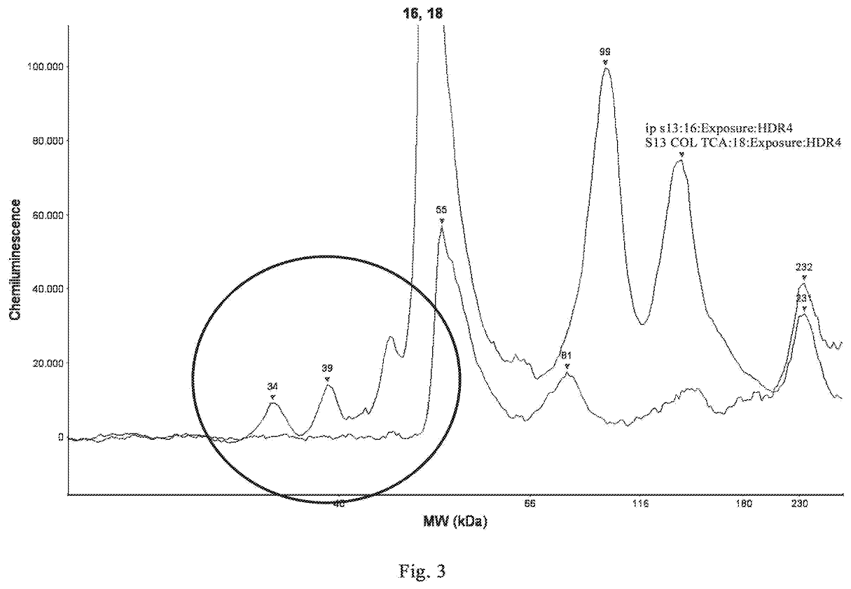

CSF was divided into two parts, one part was immunoprecipitated with the monoclonal antibody anti NGF alphaD11 (Cattaneo et al., 1988) and then processed, the other half was processed normally. The two samples were then run normally on WES. In this experiment, the peaks at 34, 40, 45 KDa have disappeared in the immunoprecipitated sample (in blue) with respect to the sample run without pretreatment (in green), thus indicating the specificity of the antibody MyBiosource. This could be for the peaks at 40 and 45 KDa of proNGF forms with post-translational modifications, as reported in the literature (Kichev et al., 2009; Pedraza et al., 2005; Pentz et al., 2020) (FIG. 3).

In order to identify the peaks without doubt, a further technique was employed: mass spectrometry.

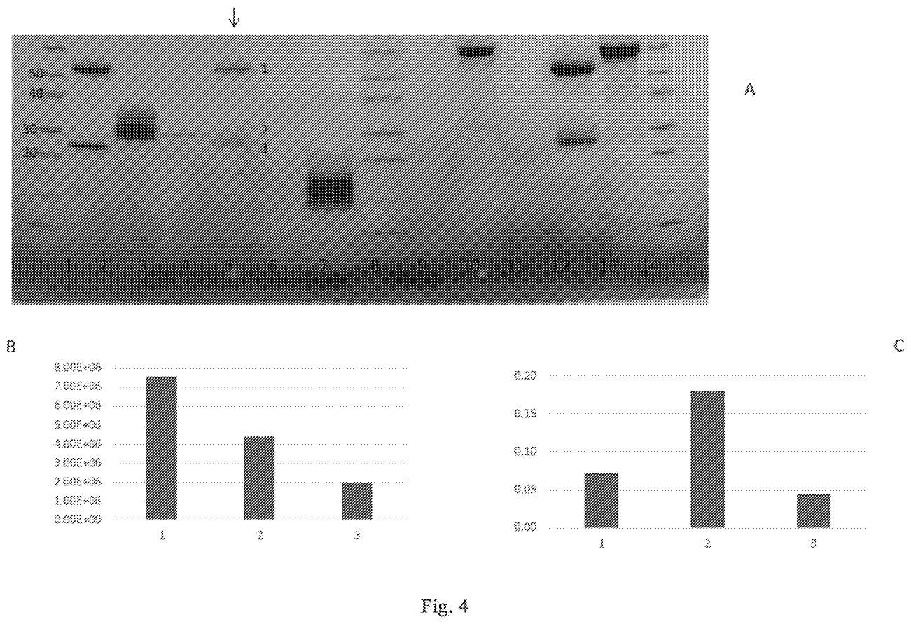

100 μl of 8 cerebrospinal fluids of patients affected by Alzheimer's Disease was joined together in order to obtain a pool. 260 μl of this pool was immunoprecipitated with the antibody Mab αD11 (Cattaneo 1988) conjugated and cross-linked in a covalent manner with the resin G Sepharose. In addition to the pool of CSF, suitable controls (10 μg recombinant NGF, 10 μg of recombinant proNGF and 3 μl of commercial human serum diluted 1:100) were immunoprecipitated with the same process. On a gel SDS-PAG with gradient 4-12% (precast BIORAD), the immunoprecipitated samples were run, alongside further non-immunoprecipitated controls (recombinant NGF and proNGF, Mab αD11). In addition to the immunoprecipitations, also the supernatants obtained from the immunoprecipitations were run on gel, containing all that which was not bonded to the antibody. The gel was colored with EZWay Protein-Quick Blue staining solution (K14050). As evident in FIG. 4A, in the range of molecular weights comprised between 60 and 2 KDa the pool of CSF from Alzheimer patients has 3 colored bands.

The mass spectrometry experiments were conducted in service by the proteomic facility of Istituto Superiore di Sanità. The Applicant supplied the facility with 2 different gels, obtained from independent experiments, from which the bands were analyzed that were present in the CSF pools from Alzheimer patients (3 bands present in 2 different pools) and the bands present in the CSF pool from patients with subjective memory disturbance (3 bands also in this pool). In all three bands, for all the pools, peptides belonging to human NGF were identified (FIGS. 4B and 4C). With independent experiments and different techniques, it was thus demonstrated that the peaks in Simple Wes along with the bands in SDS-PAGE are in fact proNGF.

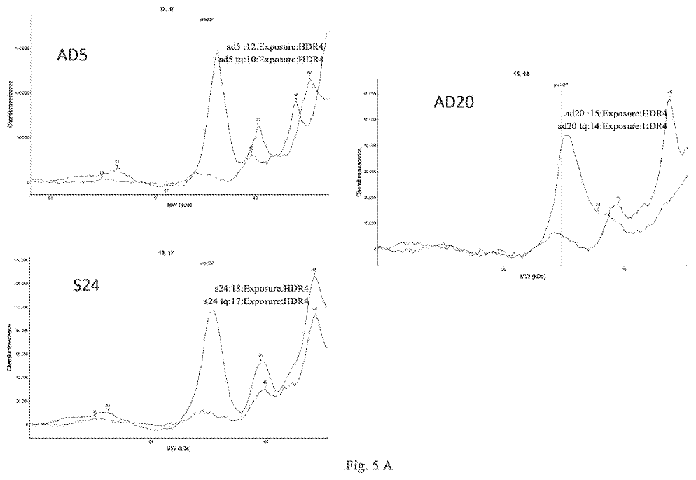

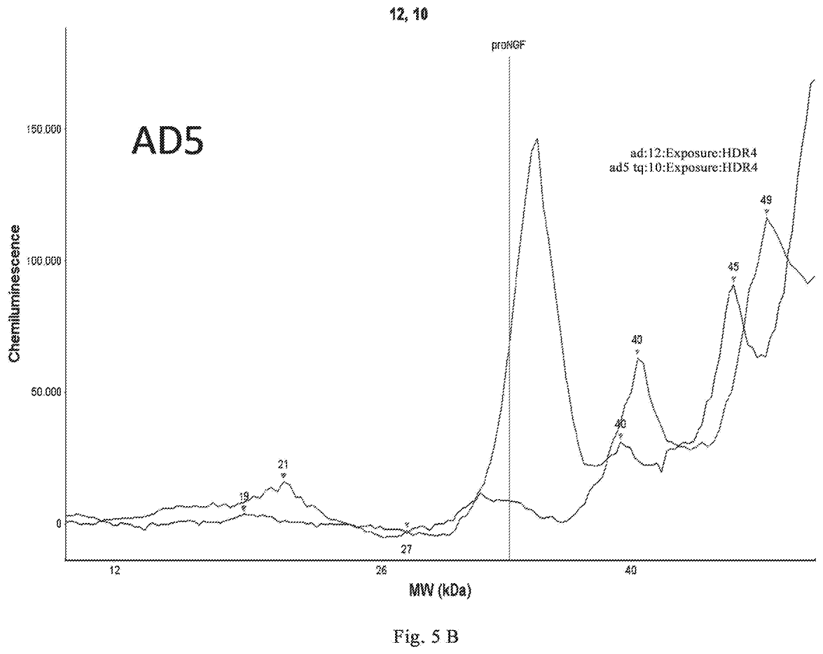

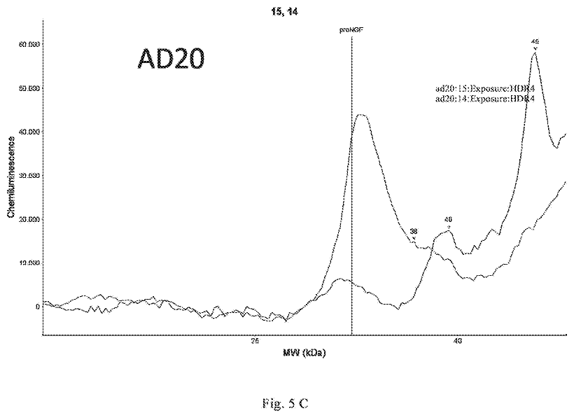

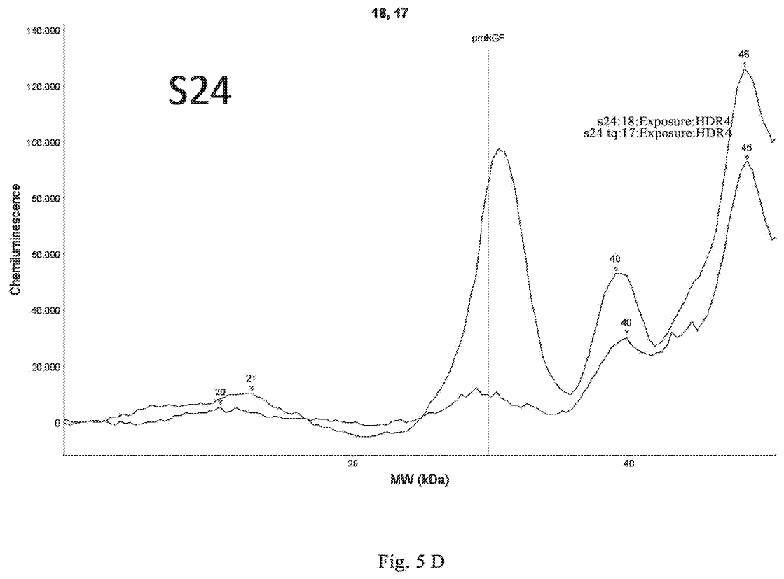

In order to understand if the method for processing the sample does not alter the concentration of proNGF, giving rise to artifacts, 3 samples of CSF were identified, characterized by a high dosage of proNGF, and newly run side-by-side with the same untreated CSF. The high dosage of proNGF ensured the possibility of seeing the peak of the precursor without having to concentrate the sample. In the samples of CSF run as is, only the peaks corresponding to the proNGF were visible and not that of NGF, as expected. (FIG. 5A). Calculating the ratio of the areas of the peak of proNGF at 34 KDa of the concentrated sample, with respect to the treated sample, the obtained ratio values, analyzed with the one sample 2-sided t-test statistical test, are around the technical value, confirming that the manipulation of the sample does not alter the concentration of proNGF. (FIGS. 5B, 5C, 5D).

TABLE 2
results relative to the analysis of the peaks of FIG. 5B

| Molecular | | area | |
|---|---|---|---|
| weight | area sample | concentrated | |
| (KDa) | as is | sample | ratio |
| 19 | 45488 | 335688.3 | 7.4 |
| 32 | 141016.5 | 1865075 | 13.2 |
| 40 | 591341 | 639629.8 | 1.1 |
| 45 | 494615.5 | 3464689 | 7.0 |

TABLE 3
results relative to the analysis of the peaks of FIG. 5C

| Molecular | | area | |
|---|---|---|---|
| weight | area sample | concentrated | |
| (KDa) | as is | sample | ratio |
| 19 | na | 100530.8 | |
| 32 | 72224.5 | 1122935 | 15.5 |
| 40 | 357204.5 | n.d. | |
| 45 | 388394 | n.d. | |

TABLE 4
results relative to the analysis of the peaks of FIG. 5D

| Molecular | | area | |
|---|---|---|---|
| weight | area sample | concentrated | |
| (KDa) | as is | sample | ratio |
| 19 | 73503 | 346305.8 | 4.7 |
| 32 | 157329.5 | 1609801 | 10.2 |
| 40 | 671722 | 1141841 | 1.7 |
| 45 | 456397.5 | 2462870 | 5.3 |

TABLE 5
(A and B): reproducibility data: measurement of
the same samples run in independent experiments
(A)

| Date of the assay | proNGF area |
|---|---|
| 10 Jul. 2019 | 318236 |
| 10 Jul. 2019 | 288567 |
| 18 Jul. 2019 | 396657 |
| 18 Jul. 2019 | 439068 |
| 9 Sep. 2019 | 477299 |
| 19 Sep. 2019 | 502616 |
| 24 Sep. 2019 | 432941 |
| 24 Sep. 2019 | 480691 |

CV % = 18.7

B

| Date of the assay | proNGF area |
|---|---|
| 18 Jul. 2019 | 847229 |
| 18 Jul. 2019 | 844174 |
| 18 Jul. 2019 | 1002126 |
| 18 Jul. 2019 | 998381 |
| 25 Jul. 2019 | 823579 |
| 25 Jul. 2019 | 815103 |
| 26 Jul. 2019 | 1180566 |
| 26 Jul. 2019 | 1170893 |

CV % = 15.8

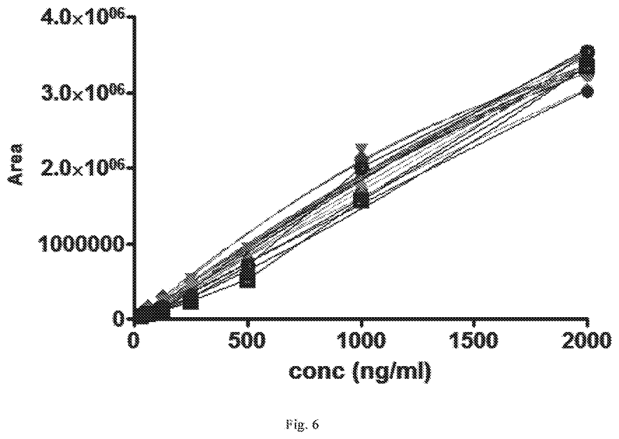

TABLE 6
results relative to calibration curves of FIG. 6

| Conc proNGF | | Standard | |
|---|---|---|---|
| (ng/ml) | Average Area | deviation | Cv % |
| 2000 | 3320156 | 3320156 | 4.2 |
| 1000 | 1990311 | 1990311 | 9.0 |
| 500 | 742869.6 | 742869.6 | 13.2 |
| 250 | 394990.5 | 394990.5 | 21.3 |
| 125 | 209406.7 | 209406.7 | 26.4 |
| 62.5 | 118064.6 | 118064.6 | 24.1 |
| 31.2 | 70445 | 70445 | 24.9 |

Analyzing the results of the biological samples, it was possible to verify the strength of the assay. The replicates rarely exceed the value of 20% for the variation coefficient, both within the same assay and in assays run on different days. Low variation coefficient values in percentage (CV %) are obtained even when the sample is processed on two different days. In order to demonstrate this, a same sample is processed and run multiple times in independent runs. The value of CV % is lower than 20%. With reference to the Tables 5 (A and B) two examples of samples of CSF have been run multiple times (S17 and S44).

Analyzing the single points of the curve, run daily for one solar month (circa 15 runs), it is possible to verify that the variation coefficients per single point remain around 20%. (FIG. 6).

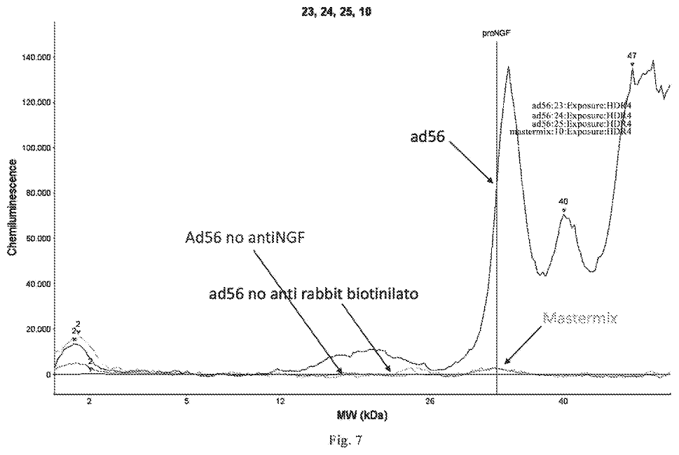

Finally, negative controls were carried out in order to evaluate the possible presence of contaminants which cross-react with the antibodies used, or a possible interference of the master mix used in the run. The following were in fact measured: 1) The mastermix without biological sample; 2) a biological sample (AD56) without primary antibody; 3) a biological sample (AD56) without secondary antibody, compared with the sample AD56 run normally. The results confirm the absence of cross-reactivity between sample and antibodies. It is instead evident that the molecular weight markers present in the master mix are recognized by the antibodies used for the assay, above all the marker of the molecular weight 2 kDa, but in a manner absolutely irrelevant for the purposes of the assay. (FIG. 7).

In order to render the inventive step of the present method appreciable, it is of interest to point out that the immunological assay designed by the inventors is the first available method capable of measuring the proNGF in a quantitative manner, by means of a calibration curve, in biological fluids, and without interference by NGF. The method is also standardized, automated and allows the processing of 24 run samples. It was demonstrated that the method is also robust, repeatable and reliable, with intra- and inter-assay results with low variation coefficient, both for the replicates of the single samples, and for the calibration curve. The sensitivity is 31 ng/ml which is greater than a western blot (circa 40 times) with respect to the same antibody in Western Blot, and is sufficient for processing samples of CSF of neurodegenerative diseases.

The volume of biological sample necessary for obtaining 5 measurements is equal to 130 microliters. With respect to that reported in the literature up to now, the automated assay thus developed by the Applicant allows measuring with calibration curve numerous samples of living patients, without wasting great quantities of biological material.

Up to now, in the course of the experiments conducted in the definition of the present invention, the following were analyzed: 84 samples of CSF of patients affected by Alzheimer's Disease, 15 CSF of people with subjected memory disturbance and 13 controls. The results of the measurements of proNGF in the three diagnostic groups were analyzed with statistical methods, and there are significant differences in the content of proNGF in the CSF between patients of Alzheimer's and subjective memory disturbance and between Alzheimer patients and controls, while there are no statistically considerable differences between subjective memory disturbance and controls.

The results in the biological samples have also detected the presence of different isoforms of proNGF, and surprisingly, in some cases, also of mature NGF. In these samples, it is therefore possible to analyze not only the proNGF, but also the relative ratio of the various isoforms of proNGF. The values of proNGF can therefore be correlated with the clinical data of the patients, allowing an improved stratification, and possibly an early diagnosis.

Finally, it is of interest to indicate that working with samples of living patients allows monitoring the value of proNGF in order to follow the progress of the disease, by means of measurements of samples drawn in different stages from the same patient, and in the case of administration of experimental therapies, allows comparing the level of proNGF in the treated patients with respect to the patients administered with placebo.

### BIBLIOGRAPHY

Acosta, C. M. R., Cortes, C., MacPhee, H., Namaka, M. P., 2013. Exploring the role of nerve growth factor in multiple sclerosis: implications in myelin repair. CNS Neurol. Disord.-Drug Targets Former. Curr. Drug Targets-CNS Neurol. Disord. 12, 1242-1256.
Belrose, J. C., Masoudi, R., Michalski, B., Fahnestock, M., 2014. Increased pro-nerve growth factor and decreased brain-derived neurotrophic factor in non-Alzheimer's disease tanopathies. Neurobiol. Aging 35, 926-933.
Capsoni, S., Brandi, R., Arisi, I., D'onofrio, M., Cattaneo, A., 2011. A dual mechanism linking NGF/proNGF imbalance and early inflammation to Alzheimer's disease neurodegeneration in the AD11 anti-NGF mouse model. CNS Neurol. Disord.-Drug Targets Former. Curr. Drug Targets-CNS Neurol. Disord. 10, 635-647.
Capsoni, S., Cattaneo, A., 2006. On the molecular basis linking Nerve Growth Factor (NGF) to Alzheimer's disease. Cell. Mol. Neurobiol. 26, 617-631.
Capsoni, S., Tiveron, C., Vignone, D., Amato, G., Cattaneo, A., 2010. Dissecting the involvement of tropomyosin-related kinase A and p75 neurotrophin receptor signaling in NGF deficit-induced neurodegeneration. Proc. Natl. Acad. Sci. 107, 12299-12304.
Cattaneo, A., Rapposelli, B., Calissano, P., 1988. Three distinct types of monoclonal antibodies after long-term immunization of rats with mouse nerve growth factor. J. Neurochem. 50, 1003-1010.
Counts, S. E., Mufson, E. J., 2005. The role of nerve growth factor receptors in cholinergic basal forebrain degeneration in prodromal Alzheimer disease. J. Neuropathol. Exp. Neurol. 64, 263-272.
Cuello, A. C., Pentz, R., Hall, H., 2019. The brain NGF metabolic pathway in health and in Alzheimer's pathology. Front. Neurosci. 13, 62.
E Counts, S., He, B., G Prout, J., Michalski, B., Farotti, L., Fahnestock, M., J Mufson, E., 2016. Cerebrospinal fluid proNGF: a putative biomarker for early Alzheimer's disease. Curr. Alzheimer Res. 13, 800-808.
Fahnestock, M., Michalski, B., Xu, B., Coughlin, M. D., 2001. The precursor pro-nerve growth factor is the predominant form of nerve growth factor in brain and is increased in Alzheimer's disease. Mol. Cell. Neurosci. 18, 210-220.
Fahnestock, M., Shekari, A., 2019. ProNGF and neurodegeneration in Alzheimer's disease. Front. Neurosci. 13, 129.
Hempstead, B. L., 2006. Dissecting the diverse actions of pro- and mature neurotrophins. Curr. Alzheimer Res. 3, 19-24.
Iulita, M. F., Cuello, A. C., 2014. Nerve growth factor metabolic dysfunction in Alzheimer's disease and Down syndrome. Trends Pharmacol. Sci. 35, 338-348.
Kichev, A., Ilieva, E. V., Pinol-Ripoll, G., Podlesniy, P., Ferrer, I., Portero-Otin, M., Pamplona, R., Espinet, C., 2009. Cell death and learning impairment in mice caused by in vitro modified pro-NGF can be related to its increased oxidative modifications in Alzheimer disease. Am. J. Pathol. 175, 2574-2585.
Malerba, F., Paoletti, F., Cattaneo, A., 2016. NGF and proNGF reciprocal interference in immunoassays: open questions, criticalities, and ways forward. Front. Mol. Neurosci. 9, 63.
Molinuevo, J. L., Ayton, S., Batrla, R., Bednar, M. M., Bittner, T., Cummings, J., Fagan, A. M., Hampel, H., Mielke, M. M., Mikulskis, A., 2018. Current state of Alzheimer's fluid biomarkers. Acta Neuropathol. (Berl.) 136, 821-853.
Paoletti, F., Covaceuszach, S., Konarev, P. V., Gonfloni, S., Malerba, F., Schwarz, E., Svergun, D. I., Cattaneo, A., Lamba, D., 2009. Intrinsic structural disorder of mouse proNGF. Proteins Struct. Funct. Bioinforma. 75, 990-1009.
Paoletti, F., Malerba, F., Kelly, G., Noinville, S., Lamba, D., Cattaneo, A., Pastore, A., 2011. Conformational plasticity of proNGF. PLOS One 6, e22615.
Pedraza, C. E., Podlesniy, P., Vidal, N., Arevalo, J. C., Lee, R., Hempstead, B., Ferrer, I., Iglesias, M., Espinet, C., 2005. Pro-NGF isolated from the human brain affected by Alzheimer's disease induces neuronal apoptosis mediated by p75NTR. Am. J. Pathol. 166, 533-543.
Pentz, R., Iulita, M. F., Ducatenzeiler, A., Bennett, D. A., Cuello, A. C., 2020. The human brain NGF metabolic pathway is impaired in the pre-clinical and clinical continuum of Alzheimers disease. Mol. Psychiatry 1-15.
Pentz, R., Iulita, M. F., Ducatenzeiler, A., Videla, L., Benejam, B., Carmona-Iragui, M., Blesa, R., Lleo, A., Fortea, J., Cuello, A. C., 2021. Nerve growth factor (NGF) pathway biomarkers in Down syndrome prior to and after the onset of clinical Alzheimer's disease: A paired CSF and plasma study. Alzheimers Dement. 17, 605-617.
Pentz, R., Iulita, M. F., Juan Fortea, A., Cuello, C., 2019. Identifying Alzheimer's disease in Down syndrome with NGF metabolism: hope for better treatment and diagnosis. T2IRS Sci Soc Bull 1-3.
Tiveron, C., Fasulo, L., Capsoni, S., Malerba, F., Marinelli, S., Paoletti, F., Piccinin, S., Scardigli, R., Amato, G., Brandi, R., 2013. ProNGF\NGF imbalance triggers learning and memory deficits, neurodegeneration and spontaneous epileptic-like discharges in transgenic mice. Cell Death Differ. 20, 1017-1030.
Xu, X.-M., Dong, M.-X., Feng, X., Liu, Y., Pan, J.-X., Jia, S.-Y., Cao, D., Wei, Y.-D., 2018. Decreased serum proNGF concentration in patients with Parkinson's disease. Neurol. Sci. 39, 91-96.
Zhang, J., Brodie, C., Li, Y., Zheng, X., Roberts, C., Lu, M., Gao, Q., Borneman, J., Savant-Bhonsale, S., Elias, S. B., 2009. Bone marrow stromal cell therapy reduces proNGF and p75 expression in mice with experimental autoimmune encephalomyelitis. J. Neurol. Sci. 279, 30-38.
Zhu, L., Pan, Q., Zhang, X.-J., Xu, Y.-M., Chu, Y., Liu, N., Lv, P., Zhang, G.-X., Kan, Q.-C., 2016. Protective effects of matrine on experimental autoimmune encephalomyelitis via regulation of ProNGF and NGF signaling. Exp. Mol. Pathol. 100, 337-343.

1. Method for the early diagnosis and the monitoring of the evolution/regression of neurodegenerative pathologies, said method providing for the quantification of a biomarker for said pathologies in a fluid that was previously drawn from a patient, said fluid being selected from among: cerebrospinal fluid; serum; urine; post mortem cerebral tissues and cellular lysates, wherein the quantified biomarker is selected from among proNGF, the latter being native proNGF; modified proNGF, the latter being proNGF in its forms with higher molecular weight, including forms with weight of 39-40 kDa and 45-50 kDa; NGF; and the proNGF/NGF ratio, said method sequentially providing for the following steps of:
Preparation of the biological sample;
Definition of the calibration curve;
Execution of run and interpolation.

2. Method for the early diagnosis and the monitoring of the evolution/regression of neurodegenerative pathologies according to claim 1, wherein the fluid previously drawn from a patient is cerebrospinal fluid and the biomarker selected is proNGF, said method providing that the preparation of the biological sample includes the concentration and desalting of the said CSF, said desalting occurring by means of disposable desalting column, said preparation of the biological sample then providing for the protein precipitation, said precipitation being executed by means of TCA, said sample then being resuspended in 0.1× Simple Wes Buffer, resulting 13 times concentrated, said sample then being admixed with the Master Mix Simple Wes and with 0.1 M DTT, boiled for 5 minutes, aliquoted and preserved at −80° C.; said method also providing that the calibration curve is defined with 8 serial dilutions (1:2) of recombinant human proNGF, that the dynamic range is 4000 ng/ml-31 ng/ml, said method providing that the run and interpolation occur by using the Simple Wes.

3. Method for the early diagnosis and the monitoring of the evolution/regression of neurodegenerative pathologies according to claim 2, wherein the running of the sample is executed on 2-40 Kda cartridges.

4. Method for the early diagnosis and the monitoring of the evolution/regression of neurodegenerative pathologies according to claim 2, wherein the CSF samples are run at least for four times, two duplicates in two different runs, said method providing that the peaks obtained as output from the Simple Wes are interpolated, said peaks having an area proportional to the concentration of the protein, said method providing that the calibration curve be interpolated with a polynomial equation of order 2, said method providing that the concentration value of the samples is obtained by interpolating the value of the area of the peak corresponding to the proNGF with the calibration curve.

5. Method for the early diagnosis and the monitoring of the evolution/regression of neurodegenerative pathologies according to claim 1, wherein the neurodegenerative pathology is selected from among Alzheimer's Disease, Down's Syndrome; Frontotemporal Dementia; Multiple Sclerosis, Amyotrophic lateral sclerosis, Parkinson's Disease and Parkinsonism, Chronic pain.

6. Method for the early diagnosis and the monitoring of the evolution/regression of neurodegenerative pathologies according to claim 5, wherein the neurodegenerative pathology is Alzheimer's Disease.

7. Method for the early diagnosis and the monitoring of the evolution/regression of neurodegenerative pathologies according to claim 5, wherein the neurodegenerative pathology is Down's Syndrome.

8. Method for the early diagnosis and the monitoring of the evolution/regression of neurodegenerative pathologies according to claim 5, wherein the neurodegenerative pathology is Frontotemporal Dementia.

9. Modified form of ProNGF, said form being proNGF with post-translational modifications, said modified form having a weight of 39-40 kDa or 45-50 KDa, for use in a method for diagnosis of neurodegenerative pathologies selected from among: Alzheimer's Disease, Down's Syndrome; Frontotemporal Dementia; Multiple Sclerosis, Amyotrophic lateral sclerosis, Parkinson's Disease and Parkinsonism, Chronic Pain.

10. ProNGF/NGF ratio for use in a method for early diagnosis of neurodegenerative pathologies selected from among: Alzheimer's Disease, Down's Syndrome; Frontotemporal Dementia; Multiple Sclerosis, Amyotrophic lateral sclerosis, Parkinson's Disease and Parkinsonism, Chronic pain, said ProNGF having a weight of 34 kDa, said NGF having a weight of 18-20 kDa.
